(19) United States
(12) Patent Application Publication
(10) Pub. No.: US 2022/0355282 A1
(43) Pub. Date: Nov. 10, 2022

(54) POROUS MATERIAL COMPOSITE COMPRISING ALLOY NANOPARTICLES, COMPOSITE CATALYST COMPRISING SAME, AND MANUFACTURING METHOD THEREFOR

(71) Applicants: Institute For Basic Science, Yuseong-gu, Daejeon (KR); Korea Advanced Institute of Science and Technology, Yuseong-gu, Daejeon (KR)

(72) Inventors: Ryong Ryoo, Yuseong-gu, Daejeon (KR); Jaeheon Kim, Yuseong-gu, Daejeon (KR); Changbum Jo, Michuhol-gu, Incheon (KR); Jeongchul Kim, Yuseong-gu, Daejeon (KR)

(73) Assignees: Institute For Basic Science, Yuseong-gu, Daejeon (KR); Korea Advanced Institute of Science and Technology, Yuseong-gu, Daejeon (KR)

(21) Appl. No.: 17/765,738

(22) PCT Filed: Sep. 29, 2020
(86) PCT No.: PCT/KR2020/013337

§ 371 Date: Mar. 31, 2022

(30) Foreign Application Priority Data
Oct. 2, 2019 (KR) 10-2019-0122612
Dec. 19, 2019 (KR) 10-2019-0171294
Sep. 28, 2020 (KR) 10-2020-0125798

(51) Int. Cl. B01J 29/74; B01J 23/63; B01J 37/02; C07C 5/333

(52) U.S. Cl. CPC B01J 29/74; B01J 23/63; B01J 37/0201; C07C 5/3337; B01J 2229/186; C07C 2529/74; C07C 2523/10; C07C 2523/63

(57) ABSTRACT

The composite according to the present invention comprises: a mesoporous inorganic support having vacancy defects; and metal alloy nanoparticles dispersed in and bound to the mesoporous inorganic support and containing a precious metal element and an earth rare element. The composite according to the present invention has a very high specific surface area since the alloy is dispersed and present in the form of ultrafine nanoparticles in the porous support, and thus can show remarkably improved activity when used as a material for a chemical reaction, including a catalyst.

### TECHNICAL FIELD

The present invention relates to a porous material composite including metal alloy nanoparticles, a composite catalyst including the same, and a manufacturing method thereof, and more particularly, to a porous material composite including intermetallic compound nanoparticles between a metal element which is difficult to reduce and a platinum-based element.

### BACKGROUND ART

An intermetallic compound has been in the spotlight recently as a catalyst material for various chemical reactions. This is due to the fact that the intermetallic compound has adsorption properties which are completely different from those of an existing single atomic compound.

A chemical reaction process which is accelerated by a catalyst includes the following processes: (1) adsorption of a reactant on a catalyst surface, (2) occurrence of reaction, and then (3) desorption of a formed material from the catalyst surface. Therefore, when adsorption specificity of a catalyst surface is changed, selectivity and activity of a chemical reaction may be also greatly changed.

In spite of the great potential as a heterogeneous catalyst, the size of the intermetallic compound is generally obtained in tens of nanometers to hundreds of nanometers, and thus, an area of a surface where the chemical reaction actually occurs is so small that an activity per unit gram is greatly deteriorated. This is because synthesis of a general intermetallic compound is performed at a very high temperature of 1000° C. or higher, so that sintering of metal particles occurs severely.

A method of synthesizing an intermetallic compound of tens of nanometers in a solution by using an organic surfactant as a structure control material has been reported (New. J. Chem., 1998, 1179-1201). According to the synthesis method, a solution containing a metal precursor, an organic surfactant, and a reducing agent is used to perform synthesis mainly at 300 to 400° C. However, the method has a limitation in that it has a low yield to have poor commerciality and the synthesized compound between dissimilar metals should be supported on a separate support. Furthermore, in order to be used as a catalyst, a structure control material which is bonded to the surface of synthesized particles should be essentially removed.

The reason why the reaction proceeds at a temperature equivalent to or higher than a melting point of a metal or synthesis is performed in a solution in the conventional synthesis method of an intermetallic compound is for guaranteeing uniform mixing at an atomic level by providing flowability to each metal. However, when a metal precursor is loaded on a porous support and then a reductive heat treatment is performed at a relatively low temperature to prepare the intermetallic compound, the metal precursor supported on the support is present in a solid state, so that the metal precursor may not have flowability in a synthesis process of a compound between dissimilar metals. Accordingly, even in the case of using a porous support having an excellent adsorption capacity such as a carbon-based support, when a compound between dissimilar metals is prepared by loading of a metal precursor and a reductive heat treatment, it is difficult for dissimilar metals which are alloyed on the support, it is substantially impossible to control the composition to be uniformly mixed and blended, and there is a high risk that a solid-solution alloy having a broad composition range, not an intermetallic compound, is manufactured. The material non-uniformity as such becomes severe when a heat treatment for removing functional groups of a metal precursor loaded on the support is performed. This is because nanoparticleization of the metal from which functional groups are removed occurs simultaneously with the removal of the functional groups. In addition, since the non-uniform distribution of the metals loaded on the support requires a longer diffusion length, a temperature rise is inevitable in a reductive heat treatment, and the size of an alloy manufactured by the high temperature is increased and becomes non-uniform. Furthermore, when the support is a non-carbonaceous material having a significantly poor adsorption capacity, the material non-uniformity becomes further severe, and most of all, as the metal to be alloyed has a high reduction potential and is difficult to reduce, a higher reduction temperature for forcing the metal to reduce is required, and thus, alloy refinement is substantially impossible.

### DISCLOSURE

### Technical Problem

An object of the present invention is to provide a composite in which metal alloy nanoparticles of a rare-earth element which is difficult to reduce are uniformly distributed on a porous support.

Another object of the present invention is to provide a composite in which intermetallic compound nanoparticles of a rare-earth element having an ultrafine size are uniformly distributed on a porous support.

Another object of the present invention is to provide a catalyst including a composite provided in the present invention.

Another object of the present invention is to provide a catalyst having significantly improved activity and/or durability.

Still another object of the present invention is to provide a manufacturing method of a composite which allows direct (in-situ) formation of metal alloy nanoparticles of a rare-earth element which is difficult to reduce on a non-carbon-based inorganic support.

### Technical Solution

In one general aspect, a composite includes: a mesoporous inorganic support having a vacancy defect, and metal alloy nanoparticles which are dispersed in and bound to the mesoporous inorganic support and include a precious metal element and a rare-earth element.

In the composite according to an exemplary embodiment of the present invention, the support may be mesoporous zeolite having a vacancy defect on a mesopore surface.

In the composite according to an exemplary embodiment of the present invention, the vacancy defect may include a silanol nest.

In the composite according to an exemplary embodiment of the present invention, a vacancy defect concentration of the support may be 0.1 mmolg⁻¹ to 1 mmolg⁻¹.

In the composite according to an exemplary embodiment of the present invention, the precious metal element may be one or two or more selected from rhodium (Rh), iridium (Ir), palladium (Pd), platinum (Pt), and ruthenium (Ru).

In the composite according to an exemplary embodiment of the present invention, the rare-earth element may have a standard reduction potential of −2.0 to −3.0 V.

In the composite according to an exemplary embodiment of the present invention, the rare-earth element may be one or two or more selected from Pr, Sc, Ho, Yb, Dy, Tm, Pm, Gd, Tb, Lu, Sm, Nd, Er, Ce, Y, and La.

In the composite according to an exemplary embodiment of the present invention, the metal alloy may be an intermetallic compound.

In the composite according to an exemplary embodiment of the present invention, the intermetallic compound may satisfy the following Chemical Formula 1:

MaₙMbₖ (Chemical Formula 1)

wherein Ma is a precious metal element, Mb is a rare-earth element, n is an integer of 1 to 7, and k is an integer of 1 to 3.

In the composite according to an exemplary embodiment of the present invention, the nanoparticles may have an average diameter of 1 to 5 nm.

In the composite according to an exemplary embodiment of the present invention, an average separation distance between the nanoparticles which are dispersed in and bound to the support may be 1 nm to 10 nm.

In the composite according to an exemplary embodiment of the present invention, the composite may include 0.5 to 5.0 wt % of the nanoparticles.

In another general aspect, a catalyst includes the composite described above.

In the catalyst according to an exemplary embodiment of the present invention, the catalyst may be a catalyst for hydrogenation, dehydrogenation, hydrodesulfurization, hydrodeoxidation, denitrification, or a steam reforming reaction.

In another general aspect, a method of dehydrogenating a saturated hydrogenation raw material using a catalyst including the composite described above is provided.

In the method according to an exemplary embodiment of the present invention, the saturated hydrocarbon raw material may include propane.

In still another general aspect, a manufacturing method of the composite described above is provided.

The manufacturing method of a composite according to the present invention includes: a) introducing a precious metal element and a rare-earth element to a mesoporous inorganic support having a vacancy defect using an impregnation method; b) subjecting the support to which a precious metal element and a rare-earth element are introduced to an oxidative heat treatment; and c) subjecting the oxidatively heat-treated support to a reductive heat treatment.

In the manufacturing method of a composite according to an exemplary embodiment of the present invention, the mesoporous inorganic support having a vacancy defect of step a) may be mesoporous zeolite from which a sacrificial element doped in a substitutional site is removed and which has a vacancy defect.

In the manufacturing method of a composite according to an exemplary embodiment of the present invention, a vacancy defect concentration may be controlled by a doping concentration of the sacrificial element.

In the manufacturing method of a composite according to an exemplary embodiment of the present invention, the sacrificial element may include one or more elements selected from aluminum, gallium, indium, and boron.

In the manufacturing method of a composite according to an exemplary embodiment of the present invention, before step c), the rare-earth element may be dispersed and positioned in the vacancy defect of the support at the atomic scale.

In the manufacturing method of a composite according to an exemplary embodiment of the present invention, the oxidative heat treatment may be performed at 300 to 500° C.

In the manufacturing method of a composite according to an exemplary embodiment of the present invention, the reductive heat treatment may be performed at 300 to 750° C. under a hydrogen flow.

In the manufacturing method of a composite according to an exemplary embodiment of the present invention, in step a), 0.5 to 3 wt % of the precious metal element and the rare-earth element may be introduced, respectively.

### Advantageous Effects

Since the composite according to the present invention includes alloy nanoparticles in which a rare-earth element which is difficult to reduce and is substantially incapable of being fine alloyed on a porous support is alloyed with a precious metal, the composite may have inherent physicochemical properties of an alloy between the rare-earth element and the precious metal. In addition, since the alloy is dispersed and present in the form of ultrafine nanoparticles in the porous support, the composite according to the present invention has a very high specific surface area, and thus, may show remarkably improved activity, when used as a material for a chemical reaction, including a catalyst.

In addition, since the composite according to the present invention is in a state in which an alloy in the form of ultrafine nanoparticles is bound to a support, it may have improved mechanical stability even in extreme environments of use.

In addition, since in the composite according to the present invention, alloy nanoparticles are dispersed in and bound to the support with a uniform size, a uniform composition, and a uniform density, the composite may show extremely homogeneous and excellent physicochemical activity in the entire region of the composite.

In addition, since in the composite according to the present invention, an intermetallic compound between the rare-earth metal having a high standard reduction potential of −1.0 V or higher, more characteristically −2.0 V or higher and the precious metal are dispersed and bound in the form of ultrafine nanoparticles of 1 to 5 nm in the support at a high density, the composite may be used in various reactions such as hydrogenation, dehydrogenation, hydrodesulfurization, hydrodeoxidation, denitrification, or a steam reforming reaction, and may be used for various uses such as a catalyst of a chemical reaction or photoreaction, an electrode material of a next-generation energy storage device including a fuel cell, a nanoelectronic material, or an optical material.

The manufacturing method of a composite according to the present invention has an advantage of directly forming an alloy between a rare-earth element which is difficult to reduce and a precious metal in the form of ultrafine nanoparticles on a non-carbon-based inorganic support.

Since the manufacturing method of a composite according to an exemplary embodiment of the present invention has no substantial restriction on a metal to be alloyed, a rare-earth-based element having a high reduction potential may be alloyed, and an intermetallic compound of a rare-earth-based element may be directly formed on an inorganic support.

The manufacturing method of a composite according to an exemplary embodiment of the present invention has an advantage of forming alloy nanoparticles with substantially the same size, the same composition, and the same density on the entire region of the support.

Since the manufacturing method of a composite according to an exemplary embodiment of the present invention is based on a traditionally well-established and simple process, which is called solid-liquid contact and heat treatment, a process is easily constructed and managed, process construction costs and production costs may be reduced, and a composite of uniform quality may be mass-produced in a short time, and thus, the method is commercially excellent.

### DESCRIPTION OF DRAWINGS

In FIG. 1, (a) is a drawing illustrating results of powder X-ray diffraction analysis of gallium-doped mesoporous zeolite (MZ-Ga), (b) is a transmission electron microscope photograph observing MZ-Ga, (c) shows results of a nitrogen adsorption-desorption test, and (d) is a drawing illustrating a Barrett-Joyner-Halenda (BJH) pore size distribution calculated from the nitrogen adsorption-desorption test results.

In FIG. 2, (a) is a drawing illustrating results of powder X-ray diffraction analysis of degalliumized mesoporous zeolite (MZ-deGa), (b) is a transmission electron microscope photograph observing MZ-deGa, (c) shows results of a nitrogen adsorption-desorption test of MZ-deGa, and (d) is a drawing illustrating a BJH pore size distribution calculated from the nitrogen adsorption-desorption test results.

In FIG. 3, (a) is a drawing illustrating results of powder X-ray diffraction analysis of mesoporous zeolite (MZ), (b) is a transmission electron microscope photograph of MZ, (c) shows results of a nitrogen adsorption-desorption test of MZ, and (d) is a drawing illustrating a BJH pore size distribution calculated from the nitrogen adsorption-desorption test results.

FIG. 4 is a drawing illustrating a spectrum of Fourier-transform infrared spectroscopy (FT-IR) of MZ, MZ-Ga, and MZ-deGa.

FIG. 5 is high-angle annular dark-field scanning transmission electron microscopy (HAADF-STEM) images observing a composite (PtY/MZ-deGa) in which Pt—Y alloy nanoparticles are formed on a MZ-deGa support, energy-dispersive X-ray spectroscopy (EDS) line profiles, fast Fourier transform (FFT) images, and a schematic diagram illustrating an atomic structure of manufactured Pt—Y alloy nanoparticles.

FIG. 6 is HAADF-STEM images observing a composite (PtLa-MZ-deGa) in which Pt—La alloy nanoparticles are formed on a MZ-deGa support, an EDS spectrum, and drawings illustrating fast Fourier transform (FFT) images.

FIG. 7 is HAADF-STEM images observing a composite (PtCe-MZ-deGa) in which Pt—Ce alloy nanoparticles are formed on a MZ-deGa support, an EDS spectrum, and drawings illustrating fast Fourier transform (FFT) images.

FIG. 8 is HAADF-STEM images observing MZ-deGa in which a non-platinum element was introduced by an incipient wetness impregnation technique and an oxidative heat treatment was performed.

FIG. 9 is a drawing illustrating a FT-IR spectrum of a sample obtained by introducing La at 1 wt %, 3 wt %, or 5 wt % to a mz-deGa support by an incipient wetness impregnation technique and then performing a heat treatment (primary heat treatment) at 350° C. for 120 minutes under an oxygen flow.

FIG. 10 is drawings illustrating results of analyzing an X-ray absorption near edge structure(XANES) of PtY-MZ-deGa, in which (a) is an XANES spectrum at a PtL₃ edge, and (b) is an XANES spectrum at a Y K edge.

FIG. 11 is a drawing illustrating results of testing propane dehydrogenation catalytic capacities of manufactured PtLa/MZ-deGa, PtY/MZ-deGa, and PtCe/MZ-deGa.

### BEST MODE

Hereinafter, referring to accompanying drawings, the composite of the present invention, a manufacturing method thereof, and a catalyst including the same will be described in detail. The drawings to be provided below are provided by way of example so that the idea of the present invention may be sufficiently transferred to a person skilled in the art to which the present invention pertains. Therefore, the present invention is not limited to the drawings provided below but may be embodied in many different forms, and the drawings suggested below may be exaggerated in order to clear the spirit of the present invention. Herein, technical terms and scientific terms used in the present specification have the general meaning understood by those skilled in the art to which the present invention pertains unless otherwise defined, and a description for the known function and configuration unnecessarily obscuring the gist of the present invention will be omitted in the following description and the accompanying drawings.

In addition, the singular form used in the specification and claims appended thereto may be intended to also include a plural form, unless otherwise indicated in the context.

In the present specification and the appended claims, the terms such as “first” and “second” are not used in a limited meaning but used for the purpose of distinguishing one constituent element from other constituent elements.

In the present specification and the appended claims, the terms such as “comprise” or “have” mean that there is a characteristic or a constituent element described in the specification, and unless otherwise particularly defined, a possibility of adding one or more other characteristics or constituent elements is not excluded in advance.

In the present specification and the appended claims, when a portion such as a film (layer), a region, and a constituent element are present on another portion, not only a case in which the portion is in contact with and directly on another portion but also a case in which other films (layers), other regions, other constitutional elements are interposed between the portions is included.

The composite according to the present invention includes: a mesoporous inorganic support having a vacancy defect; and metal alloy nanoparticles which are dispersed in and bound to the mesoporous inorganic support and include a precious metal element and a rare-earth element.

According to IUPAC definition, the mesoporous inorganic support may refer to an inorganic support having pores having a diameter of 2 nm to 50 nm. However, the mesoporous inorganic support should not be interpreted as an inorganic support having no micropores (pores of less than 2 nm) or macropores (pores of more than 50 nm) (excluding the micropores or macropores), and should be interpreted as an inorganic support having at least mesopores, of course.

Specifically, the mesoporous inorganic support may refer to a porous inorganic support including mesopores and having a specific surface area of 200 to 1000 m²/g. In addition, the mesoporous inorganic support may refer to an inorganic support having a volume by mesopores per unit mass of the support of at least 0.2 ml/g or more, advantageously 0.5 ml/g or more, more advantageously 0.8 ml/g or more, and still more advantageously 1.0 ml/g or more. In addition, the mesoporous inorganic support may refer to an inorganic support having at least one or more, specifically one, two, or three or more peaks positioned at 2 nm to 50 nm, based on a pore size distribution. Here, the specific surface area, the pore volume, the pore distribution, and the like of the porous inorganic support may be calculated experimentally, by interpreting the data of an adsorption amount to a relative pressure using an argon or nitrogen gas adsorption method (argon or nitrogen gas isothermal adsorption/desorption curve) according to BET or non-local density functional theory (NLDFT), of course.

Pores in a mesoporosity range are advantageous, since they may provide a large specific surface area (specific surface area of a support) and nanoparticles may be homogeneously manufactured on a surface (pore surface) resulting from an open porous structure as well as on an outer surface of an inorganic support. That is, a mesoporous inorganic support having a vacancy defect allows manufacture of nanoparticles having substantially the same size and distribution on the outer surface of an inorganic support and the surface resulting from porosity.

An inorganic support may be amorphous, crystalline, or a mixed phase of amorphous and crystalline phases. In an advantageous example, the inorganic support may be crystalline or a mixed phase of crystalline and amorphous phases, and more advantageously, the inorganic support may be crystalline. The crystalline inorganic support may refer to a monocrystalline or polycrystalline inorganic support. The crystalline inorganic support is more effective for use as a catalyst, since it has improved thermal/hydrothermal stability.

When the inorganic support is crystalline, porosity including mesopores may be provided by a skeletal structure of an inorganic compound forming the inorganic support. As a specific example, when the inorganic support is a mesoporous crystalline support, it may have a pore structure in which mesopore channels are two-dimensionally or three-dimensionally, regularly or irregularly arranged. In this case, nanoparticles may be dispersed in and bound to the surface including the channel wall (pore surface) of the mesopore channel as well as the outer surface of the inorganic support.

The lattice defect of the mesoporous inorganic support allows alloying of a rare-earth element which has a high reduction potential so that fine alloying is accepted as being substantially impossible in-situ on a support. That is, a rare-earth metal which is extremely difficult to reduce may be fine alloyed in-situ on the support by the lattice defect of the mesoporous inorganic support.

Specifically, in order to directly form alloy-type nanoparticles on a support, firstly, each metal should be introduced to the support using the precursor (metal source) of each metal forming the alloy. As is known, when the support is a non-carbon-based inorganic support, the adsorption capacity of the support itself is deteriorated so that it is difficult to adsorb the metal source in a homogeneously mixed state, and when a pretreatment for removing a ligand is performed, agglomeration of metal(s) occurs by energy applied in a pretreatment step, and the inhomogeneity is increased. That is, even in the case in which a support is impregnated in a solution containing a metal precursor and a metal source(s) is(are) loaded as homogeneously as possible on a support, metal(s) is(are) individually agglomerated by energy applied in a pretreatment step for removing ligand and the like. By the limitations, relatively coarse particles are produced, and nanoparticles are likely to be manufactured in the form of a solid solution having an inhomogeneous composition. When the heat treatment is performed at a high temperature for compensating for the inhomogeneous state, not only the size but also the size distribution of the nanoparticles is increased. Furthermore, when an intermetallic compound of a metal having a high standard reduction potential such as a rare-earth element is targeted, a metal which is difficult to reduce should be reduced, and thus, the heat treatment should be performed at a higher temperature (usually 1000° C. or higher), and it is substantially impossible to form the intermetallic compound into an ultrafine nanoparticle phase at a high density.

However, when the inorganic support is a mesoporous inorganic support having a vacancy defect, the rare-earth element which is difficult to reduce may be homogeneously introduced to a single atomic unit by the vacancy defect of the inorganic support, and agglomeration of metal may be prevented even after the pretreatment for removing ligand.

In particular, the support may be mesoporous zeolite having a vacancy defect on the surface of mesopores, and in this case, the vacancy defect may include a silanol nest. The silanol nest allows the rare-earth element to be distributed on the support as a single atomic species, and also, allows reduction of the rare-earth element even at a low reductive heat treatment by activation of the rare-earth element having a high standard reduction potential.

Thus, the mesoporous zeolite support having a vacancy defect including the silanol nest formed allows ultrafine alloying into an average size of 1 to 5 nm in-situ on the support, even when a rare-earth element, furthermore, a rare-earth element having a standard reduction potential of −1.0 to −3.0 V, and more characteristically a rare-earth element having a standard reduction potential of −2.0 to −3.0 V are used.

By the reasons described above, the support may be advantageously a mesoporous crystalline inorganic support having a vacancy defect, and more advantageously, mesoporous zeolite having a vacancy defect, substantially a vacancy defect including a silanol nest on the surface of mesopores. The mesoporous zeolite may form a structural defect including a silanol nest homogeneously in very large amount in a skeleton, by removing a doping element (sacrificial element) or adjusting synthesis conditions. In the term zeolite, the zeolite may be interpreted as including a material including silica and optionally alumina. However, it should be recognized that silica and alumina portions may be entirely or partially replaced with other oxides, which is well-known to a person skilled in the art. As an example, the silica portion may be replaced with germanium oxide, tin oxide, phosphorus oxide, and the like. The alumina portion may be replaced with boron oxide, iron oxide, gallium oxide, indium oxide, and the like. Therefore, the term zeolite should be interpreted as including not only a material containing silicon and optionally an aluminum atom in a crystalline lattice structure, but also a material containing a replacement atom appropriate for silicon and aluminum, such as gallosilicate, silicoaluminophosphate (SAPO), and aluminophosphate (ALPO).

By a mesoporous zeolite support having a vacancy defect including a silanol nest formed, alloy nanoparticles between a rare-earth element and a precious metal element which are ultrafine and have a uniform size may be formed in a manner of being substantially homogeneously dispersed in and bound to the entire region of an inorganic support, and intermetallic compound nanoparticles between a rare-earth element and a precious metal element may be formed.

By the methodological characteristic of introducing a rare-earth element to a support and activating it as a single atomic species by the vacancy defect including a silanol nest, the support may include the vacancy defect again in a state in which the nanoparticles are produced directly (in-situ) on a support. Thus, in the composite, the inorganic support including a vacancy defect, in particular, a silanol nest may correspond to an indicator showing a methodological characteristic.

Though the size and the specific shape of the inorganic support may be properly adjusted considering the use of the composite, the mesoporous inorganic support may be particulate, specifically monocrystalline or polycrystalline particulate, and may have a size in the order of tens of nanometers to several micrometers. Here, in the case of the mesoporous zeolite support including a vacancy defect in which mesopores are formed by a chemical skeleton and a silanol nest is included on a mesopore surface by a skeleton defect, even when the support is coarse particulate having a size (diameter) up to several micrometers (μm), the ultrafine intermetallic compound nanoparticles between a rare-earth element and a precious metal may be homogeneously distributed in the entire region of the support.

Here, the vacancy defect of the inorganic support is different from the vacancy defect which is naturally present in the inorganic support by a thermal equilibrium state according to thermal history in the manufacturing process of the inorganic support. It is clear that the vacancy defect of the inorganic support means an excessive amount of vacancy defect as compared with the vacancy defect produced by the thermal equilibrium state, and means an artificially formed vacancy defect, of course.

As an example of an artificially formed vacancy defect level beyond the thermal equilibrium state, the inorganic support may include a vacancy defect at a concentration of 0.1 mmolg⁻¹ to 1.0 mmolg⁻¹, and more specifically, the mesoporous zeolite may include a silanol nest at a concentration of 0.1 mmolg⁻¹ to 1 mmolg⁻¹. The defect concentration as such is a concentration which is clearly distinguished from a concentration in a thermal equilibrium state, and furthermore, is a concentration at which substantially all rare-earth elements introduced to the support may be distributed as a single atomic species by a defect such as a silanol nest without damaging (collapsing) the crystal structure of the support, and is a concentration at which, when a reductive heat treatment is performed for alloying, atomically reduced rare earth may be homogeneously supplied to each particulate precious metal.

As an example of the artificially formed vacancy defect beyond the thermal equilibrium state, the inorganic support included in the composite may include a vacancy defect by removal of a sacrificial element doped in a substitutional site. That is, the inorganic support may include a vacancy defect produced by an artificial process in which a doping element is doped in the substitutional site of the inorganic support by a step of manufacturing a support; or an independent step after manufacturing a support, and then the doped element (sacrificial element) is removed. Accordingly, the vacancy defect concentration of the composite may be directly controlled by the doping concentration of the sacrificial element.

In the metal alloy nanoparticles, the metal alloy may include binary metal nanoparticles, ternary metal nanoparticles, quaternary, or more nanoparticles.

In the metal alloy nanoparticles, the alloy may include a solid solution or intermetallic compound. A solid solution may refer to a structure in which each of two or more metals randomly occupies a lattice site while a single crystal structure is maintained, or a structure in which one element (metal) is randomly solid-solubilized and present in a substitutional or interstitial manner in the crystal structure of another element (another metal). The intermetallic compound is clearly distinguished from the solid solution. The intermetallic compound may refer to a thermodynamically stable or meta-stable, substantially thermodynamically stable compound in which elements (metals) forming the compound are bonded in an integer ratio, a long-range order is shown, and the atom of each metal occupies a certain position in the crystal lattice to form a unique crystal structure. The composition, the crystal structure, and the like of the intermetallic compound are well known to a person skilled in the art by a phase diagram of elements forming the alloy.

The alloy nanoparticles may be crystalline particles, specifically polycrystalline or monocrystalline particles, and more specifically monocrystalline particles.

Binding between the nanoparticles and the support may refer to direct binding of the nanoparticles to the support and physical (mechanical) integration of the nanoparticles with the support. The binding is different from fixation by van der Waals interaction which may be formed when introducing the previously manufactured nanoparticles to the support, or fixation by a functional group (support and/or nanoparticles).

Specifically, the binding between the nanoparticles and the support may refer to a state in which the nanoparticles are bonded and attached to the support while forming a two-dimensional interphase interface between the nanoparticles and the support. In the manufacturing method, the binding between the nanoparticles and the support may be formed as the nanoparticles are nucleated and grows on the support.

The nanoparticles may have an ultrafine average size (diameter) of 1 to 5 nm, and may have a significantly uniform size distribution. As the size of the nanoparticles dispersed in and bound to the support is small and uniform, the physicochemical properties of the composite including catalytic activity may be improved and the physicochemical properties may be shown uniformly, which is thus, advantageous. Specifically, the alloy nanoparticles dispersed in the support may have an average diameter of 1 to 5 nm, more specifically 1 to 4 nm, and still more specifically 1 to 3.5 nm. Here, since the nanoparticles are extremely fine, the size (diameter) of the nanoparticles, the separation distance between the nanoparticles, and the like may be measured by a transmission electron microscope (TEM) or a scanning-transmission electron microscope (STEM), and the average value may be measured by observing the statistically significant number of nanoparticles. The statistically significant number of nanoparticles may refer to 100 or more, substantially 300 or more, but is not limited thereto. In addition, the average size of the nanoparticles may be calculated as a converted size (diameter of a circle) obtained by converting the nanoparticles into circles having the same size of the nanoparticles, independently of the shape of the nanoparticles. In addition, the separation distance between the nanoparticles may refer to a straight-line distance between one nanoparticle and an adjacent nanoparticle thereto, based on one nanoparticle, in an image observed by a transmission electron microscope (TEM) or a scanning-transmission electron microscope (STEM), of course. In addition to the measurement method using an electron microscope, the size and the dispersion degree of the nanoparticles may be measured by a hydrogen adsorption method.

As described above, the composite may include the alloy nanoparticles between the rare-earth element and the precious metal element, which are extremely fine and have a uniform size, and furthermore, may include the alloy nanoparticles which are uniformly spaced apart and dispersed with a high density and bonded to a support. The refinement, densification, and uniform dispersibility of the nanoparticles increase the specific surface area of an alloy (surface area per alloy in a unit weight) and also increase the active site participating the reaction, and thus, are very effective for catalytic application. Specifically, the nanoparticles may be dispersed in and bound to the support with an average separation distance between the nanoparticles of 1 to 10 nm, specifically 1 to nm while having an ultrafine size. Considering the average size of the nanoparticles described above, the average separation distance as such may substantially correspond to the average size of the intermetallic compound.

The composite may have the inherent physicochemical properties of the alloy, and since the support is a non-carbon-based inorganic support, thermal, mechanical, and chemical stability may be secured, and furthermore, since the alloy nanoparticles are in the state of being bound to the support, the composite may have further improved thermal and mechanical stability. In addition, since the alloy is homogeneously bound in the form of ultrafine nanoparticles to the mesoporous support, the composite may have very uniform and high reactivity (activity). In addition, since the alloy is uniformly dispersed at a high density in the form of ultrafine nanoparticles, the content (supported amount) of the alloy required for securing the previously designed activity of the composite may be decreased. The fact that previously designed activity may be secured by a small amount of the alloy means that the amount of the precious metal used of which the resource is difficult to secure may be decreased, and the production costs of the composite may be lowered. In addition, it may be used for various applications, only by simple design modification, which is to design an alloy material considering the previously known physicochemical properties of the alloy, together with its excellent commerciality.

As described above, the rare-earth element which is difficult to reduce may be alloyed by the vacancy defect, in particular, the silanol nest included in the inorganic support.

As is known, the rare-earth element having a high reduction potential is difficult to reduce, and in order to alloy the rare-earth element, very high energy (such as thermal energy, light energy, chemical energy, and/or electrical energy) should be applied. It is known that by the limitation, it is very difficult to fine-particleize (independent particleization of the support) the rare-earth element having a high standard reduction potential to a several nanometer level, and furthermore, a technique of ultrafine alloying of 5 nm or less in a state of being directly bound to the support has not been reported.

Thus, the composite according to a characteristic exemplary embodiment of the present invention may include alloy nanoparticles between the rare-earth element and the precious metal element, which have an average size of 1 to 5 nm and are dispersed in and bound to the support, and more characteristically, may include alloy nanoparticles between the rare-earth element having a standard reduction potential of −1.0 to −3.0 V and the precious metal element, and more characteristically, alloy nanoparticles between the rare-earth element having a standard reduction potential of −2.0 to −3.0 V and the precious metal element.

The composite according to a more characteristic exemplary embodiment of the present invention may include intermetallic compound nanoparticles between the rare-earth element and the precious metal element, which have an average size of 1 to 5 nm and are dispersed in and bound to the support, and more characteristically, may include intermetallic compound nanoparticles between the rare-earth element having a standard reduction potential of −1.0 to −3.0 V and the precious metal element, and more characteristically, intermetallic compound nanoparticles between the rare-earth element having a standard reduction potential of −2.0 to −3.0 V and the precious metal element.

Here, the standard reduction potential is the standard reduction potential (unit: V) according to the following Reaction Formula 1, of course, and is based on the potential value (0 V) of a standard hydrogen electrode, of course:

Mⁿ⁺+ne⁻→M (Reaction Formula 1)

wherein Mⁿ⁺ is an ion of a rare-earth element having an oxidation state of n, e⁻ is an electron, and M is a neutral solid rare-earth element. Here, n may be a natural number of 1 to 4. In Reaction Formula 1, when the rare-earth element has a known oxidation number of two or more, the standard reduction potential of Reaction Formula 1 may be a standard reduction potential based on an oxidation number in a most thermodynamically stable state (conditions of room temperature and normal pressure), among known various oxidation numbers of the corresponding rare-earth element.

The precious metal element may be properly selected considering the known physicochemical properties of a spherical alloy (solid solution or intermetallic compound) and the use of the composite. As a specific example, the precious metal element may be a platinum group element, and the platinum group element (first metal) may be one or two or more selected from rhodium (Rh), iridium (Ir), palladium (Pd), platinum (Pt), and ruthenium (Ru), but is not necessarily limited thereto.

The rare-earth element may be one or two or more selected from the group consisting of Sc, Y, La, Ce, Pr, Nd, Pm, Sm, Eu, Gd, Tb, Dy, Ho, Er, Tm, Yb, and Lu. Characteristically, an example of the rare-earth element having a standard reduction potential of −1.0 to −3.0 V may include one or two or more elements selected from Eu, Pr, Sc, Ho, Yb, Dy, Tm, Pm, Gd, Tb, Lu, Sm, Nd, Er, Ce, Y, and La. More characteristically, an example of the rare-earth element having a standard reduction potential of −2.0 V to −3.0 V may include one or two or more elements selected from Pr, Sc, Ho, Yb, Dy, Tm, Pm, Gd, Tb, Lu, Sm, Nd, Er, Ce, Y, and La.

In the composite according to an exemplary embodiment of the present invention, the alloy nanoparticles may be intermetallic compound nanoparticles, and the intermetallic compound may satisfy the following Chemical Formula 1:

MaₙMbₖ (Chemical Formula 1)

wherein Ma is a precious metal element, specifically a platinum group element, Mb is a rare-earth element, characteristically a rare-earth element having a standard reduction potential of −1.0 to −3.0 V, more characteristically a rare-earth element having a standard reduction potential of −2.0 to −3.0 V, n is an integer of 1 to 7, and k is an integer of 1 to 3.

A substantial example of the intermetallic compound may include an intermetallic compound of Ma3Mb1 such as Pt₃La, Pt₃Y, Pt₃Gd, Pt₃Ce, Pt₃Sc, Pd₃La, Pd₃Ce, or Pd₃Y, but is not necessarily limited thereto.

In a specific example, the composite may include 0.5 to 5.0 wt %, specifically 0.5 to 3 wt %, and more specifically 0.5 to 1.5 wt % of the alloy nanoparticles, but is not necessarily limited thereto.

The present invention includes a catalyst including the catalyst described above.

The composite described above may have the following characteristics: physicochemical properties which are imparted by an intermetallic compound between a precious metal and a rare-earth element or a solid solution alloy itself between a precious metal and a rare-earth element, having a homogeneous composition; an alloy metal being dispersed in and bound to a support in the form of ultrafine nanoparticles; multinary metal being mechanically stably bound to a support while forming a two-dimensional interface with the support; the support being a non-carbon-based inorganic support; the multinary metal being uniformly dispersed and bound at an extremely high density; mesoporosity providing a stable material movement path which secures smooth flow and a contact even in the case of a raw material (reaction material) having a high molecular weight; alloy nanoparticles being evenly dispersed in and bound to the entire region of the support ranging from the surface to the center of the support, in spite of a very large specific surface area; and the alloy nanoparticles being dispersed in and bound to the entire region of the support ranging from the surface to the center of the support with the same size and distribution as the surface. Thus, the composite may have an excellent catalytic capacity and improved durability.

Since the catalytic action of the composite is imparted by an alloy between a precious metal and a rare-earth element, the composite may be used as previously known various reaction catalysts for each alloy. As a specific example, as a platinum-based catalyst or a multinary metal-based catalyst including a platinum-based material is known as a catalyst for hydrogenation, dehydrogenation, hydrodesulfurization, hydrodeoxidation, denitrification, or a steam reforming reaction, according to an exemplary embodiment of the present invention, the catalyst including the composite described above may be a catalyst for oxidation, hydrogenation, dehydrogenation, hydrodesulfurization, hydrodeoxidation, denitrification, a steam reforming reaction, or a methanol steam reforming reaction. That is, the catalyst according to an exemplary embodiment of the present invention may have a previously known catalytic capacity depending on the inherent physical properties of the alloy included in the composite.

Thus, the present invention includes an oxidation method, a hydrogenation method, a dehydrogenation method, a hydrodesulfurization method, a hydrodeoxidation method, a denitrification method, a steam reforming method, or a methanol steam reforming method.

As a characteristic example of the catalyst according to the present invention, when the catalyst (composite) is an alloy between Pt and a rare-earth element, in particular, an intermetallic compound, the catalyst may be a catalyst for dehydrogenation reaction of a raw material including a saturated hydrocarbon raw material, more characteristically, propane.

Specifically, the catalyst according to an exemplary embodiment of the present invention may include a non-carbon-based mesoporous inorganic support including a vacancy defect, and nanoparticles which have an average size of 1 to 5 nm, are dispersed in and bound to the support, and are an intermetallic compound between Pt and a rare-earth element, and the catalyst may be a catalyst for dehydrogenation reaction of a saturated hydrocarbon raw material, more characteristically a raw material including propane.

When the catalyst includes the intermetallic compound between Pt and a rare-earth element, a propane conversion rate and propylene selectivity which are significantly improved for a long time of 30 days or more may be shown even under harsh conditions in which a commercially used PtSn/alumina catalyst lose catalytic activity in one day.

More specifically, the dehydrogenation method according to an exemplary embodiment of the present invention may include loading a pelletized composite on a common fixed-catalyst bed device, and then supplying a raw material gas including propane under typical propane dehydrogenation reaction conditions, specifically at a temperature of 525 to 705° C. under a pressure of 1 to 3 bar at a propane weight space velocity of 0.1 to 10 h⁻¹. The raw material gas may further include one or more gaseous phases selected from nitrogen, hydrogen, and water vapor, and the amount may be 30 vol % to 80 vol %. Here, before a dehydrogenation reaction, the catalyst loaded on a fixed catalyst bed device is activated under usual conditions (as an example, hydrogen atmosphere and reaction temperature) and then a dehydrogenation reaction may be performed, of course.

The present invention includes a manufacturing method of the composite described above. In the manufacturing method according to an exemplary embodiment of the present invention, the support includes all descriptions related to the inorganic support described above based on the composite described above. In the manufacturing method according to an exemplary embodiment of the present invention, a rare-earth element, a precious metal element, and alloy nanoparticles include the descriptions related to the rare-earth element, the precious metal element, and the alloy nanoparticles described above based on the composite.

The manufacturing method of a composite according to the present invention includes: a) introducing a precious metal element and a rare-earth element to a mesoporous inorganic support having a vacancy defect using an impregnation method; b) subjecting the support to which a precious metal element and a rare-earth element are introduced to an oxidative heat treatment; and c) subjecting the oxidatively heat-treated support to a reductive heat treatment.

Since the manufacturing method according to the present invention does not use an organic or inorganic template, it is a manufacturing method which does not require a post-treatment such as template removal, since ultrafine alloy nanoparticles are directly formed on an inorganic support, it is a method of performing both synthesis of particles and loading on a support, it is a method capable of forming an alloy between a rare-earth element which is extremely difficult to reduce and a precious metal in-situ on a support, and it is a method capable of physically (mechanically) stably and evenly dispersing and bounding an intermetallic compound or a solid solution alloy in the form of ultrafine nanoparticles having an average size of only 1 to 5 nm in/to a support.

An impregnation method is a method of introducing a metal to a support by applying or permeating a precursor solution in which a precursor of a metal to be desired is dissolved on a support, and specifically, the impregnation method of step a) may be an incipient wetness impregnation technique. As is known, the incipient wetness impregnation technique is very advantageous for introducing a metal to a support at a designed concentration (wt %).

Substantially, step a) may be a step of introducing a precious metal element and a rare-earth element to a mesoporous support having a vacancy defect by the incipient wetness impregnation technique using a precursor solution including the precursor of the precious metal element (hereinafter, a first precursor) and the precursor of the rare-earth element (hereinafter, a second precursor).

The first precursor and the second precursor may be independently of each other formed of a cation and an anion ligand including the precious metal element or the rare-earth element. The cation may be a precious metal ion or a rare-earth ion itself, but is not limited thereto, and may include a cation of a metal (precious metal or rare earth) to which an organic ligand such as NH₃ is coordinated. The anion ligand is easily removed in a gaseous phase at the time of an oxidative heat treatment, and may be a ligand having a negative charge. The anion ligand which is easily removed in a gaseous phase at the time of an oxidative heat treatment may include an anion organic ligand, and the anion organic ligand may include ligands formed of elements such as C, N, O, H, I, Br, and Cl, as is known. As a substantial example, the anion ligand may include NO₃⁻, NO₂⁻, OH—, and the like, but the present invention may not be limited by the specific material of the first precursor or the second precursor, of course.

A solvent of the precursor solution may be any liquid material which easily dissolves the first precursor and the second precursor and does not react with the support, but since the support is an inorganic support, the solvent may be any material as long as it is a liquid material which substantially dissolves the precursor. As a specific and non-limiting example, the solvent of the precursor solution may be a polar solvent, and the polar solvent may include methanol, isobutanol, ethanol, 1-propanol, 2-propanol, 1-butanol, 2-methoxyethanol, diethylethanolamine, ethylenediamine, γ-butyrolactone, ethylene glycol, diethylene glycol, diethylenetriamine, N-methylethanolamine, N-methyl-2-pyrrolidone, N,N-dimethylformamide, dimethylsulfoxide, water, or a mixed solvent thereof, but the present invention is not limited thereto. Since the volume of the precursor solution applied in the incipient wetness impregnation technique is predetermined, the concentration of the precursor (first precursor or second precursor) of the precursor solution may be properly changed considering the amounts of the precious metal element and the rare-earth element which is to be introduced to the support. As a substantial example, the precursor solution may include 0.1 to 20 wt % of the precursor (first precursor or second precursor), but the present invention is not limited thereto. As a substantial example, 0.5 to 3 wt %, specifically 0.5 to 2 wt %, and more specifically 0.5 to 1.5 wt % of each of the precious metal element and the rare-earth element may be introduced to the support by the impregnation method of step a), but the present invention is not necessarily limited thereto.

Any method may be used for applying the precursor solution as long as it is a method capable of adsorbing the precursor in the liquid on the inorganic support. As a substantial and non-limiting example, in step a), a contact between the inorganic support and the precursor solution may be performed by spraying, dipping, supporting, and the like. Specifically, the contact between the precursor solution and the inorganic support may be performed by spraying the precursor solution on the inorganic support, dipping the inorganic support in the precursor solution, or supporting the precursor solution on the inorganic support. Here, a step of recovering the dipped inorganic support after dipping may be further performed, of course, and drying of the inorganic support in contact with the precursor solution may be further performed, of course. However, since drying of the inorganic support may be simultaneously performed in a temperature raising process for a heat treatment, drying of the inorganic support may be selectively performed, if necessary, of course.

The heat treatment (first heat treatment) of step b) may be a heat treatment for removing an organic ligand of a metal precursor (first precursor or second precursor) in a gaseous phase. Accordingly, the heat treatment of step b) may be performed in an oxidizing atmosphere, that is, in an atmosphere containing oxygen. Since it is advantageous that the organic ligand is removed within a short time, the oxidizing heat treatment of step b) may be performed in an atmosphere where pure oxygen gas or inert gas containing at least 10 vol % of oxygen flows, as an example, under 300 to 800 cm³min⁻¹g_{cat}⁻¹ per unit mass of the support. A heat treatment temperature in the heat treatment of step b) (first heat treatment) may be a temperature at which the organic ligand is removed well in a gaseous phase under an oxidizing atmosphere. However, in terms of suppressing agglomeration of a metal remaining after removing the organic ligand, it is advantageous to perform the heat treatment at a temperature of 500° C. or lower. As a substantial and non-limiting example, the oxidative heat treatment may be performed at 300 to 500° C., more substantially 300 to 450° C. An oxidative heat treatment (first heat treatment) time may be 60 to 480 minutes, but the present invention may not be limited by the conditions of the heat treatment for ligand removal (oxygen content in the atmosphere, temperature, time, and the like), of course.

The alloy nanoparticles between the rare-earth element and the precious metal element on the support may be directly formed by the reductive heat treatment (second heat treatment).

In a characteristic example according to the manufacturing method of the present invention, the inorganic support of step a) may be a mesoporous inorganic support including a vacancy defect, advantageously a mesoporous zeolite including a silanol nest.

By inserting the rare-earth element having a high standard reduction potential (that is, having a more negative standard reduction potential) into a silanol nest as a single atomic species, the rare-earth element may be dispersed and positioned in the support at an atomic level, is easily reduced at a low reductive heat treatment temperature by activation by the silanol nest, and allows uniform supply of a material ion an atomic unit at the time of the reductive heat treatment.

In addition, a mesoporous inorganic support having a large amount of structural defects formed, advantageously mesoporous zeolite having a large amount of silanol nest allows a metal source to easily penetrate into the inorganic support through a mesopore channel, and furthermore, the rare-earth element is distributed as a single atomic species evenly in the entire region of the mesoporous support including a pore wall (pore surface) so that alloy nanoparticles having substantially the same size and density may be manufactured on the outer surface of the inorganic support and inside the inorganic support, and ultrahigh-density alloy nanoparticles may be manufactured.

The rare-earth element is inserted into the silanol nest and uniformly dispersed in an atomic unit, and also, the agglomerate (cluster) of the precious metal which may be produced in step b) is reduced by itself in step c) and simultaneously all agglomerates may be supplied isodirectionally and uniformly with the rare-earth element (reduced rare-earth element) and converted into an intermetallic compound or a solid solution.

Furthermore, when the rare-earth element is inserted into the silanol nest and atomically dispersed, at the time of the reductive heat treatment of step c), the rare-earth element inserted in an atomic unit into the defect may diffuse well with the help of the defect, and thus, ultrafine nanoparticles having a uniform composition, in particular, in the form of an intermetallic compound may be synthesized.

The reductive heat treatment of step c) may be performed in an atmosphere where pure hydrogen gas or inert gas containing at least 10 vol % of hydrogen flows, as an example, under 300 to 1000 cm³min⁻¹g_{cat}⁻¹ per unit mass of the support. A heat treatment temperature in the reductive heat treatment (second heat treatment) of step c) may be 300° C. to 750° C., specifically 550° C. to 700° C. so that the rare-earth element is reduced well with the help of the silanol nest and fine alloying is possible. A reductive heat treatment (second heat treatment) time may be 60 to 480 minutes, but is not limited thereto.

As described above, in the manufacturing method according to an advantageous exemplary embodiment of the present invention, the inorganic support in contact with the precursor solution in step a) may be a mesoporous inorganic support having a vacancy defect formed, advantageously, crystalline mesoporous zeolite having a vacancy defect formed, and more advantageously, crystalline mesoporous zeolite having a vacancy defect including a silanol nest formed. More specifically, the support may have a vacancy defect, specifically a silanol nest formed at a concentration of 0.1 mmolg⁻¹ to 1.0 mmolg⁻¹.

The inorganic support having a vacancy defect formed may be manufactured by any method which may form the vacancy defect in the crystalline inorganic support. As an example, the vacancy defect may be formed by doping a sacrificial element doped in a substitutional site and then removing the doped sacrificial element, in the step of synthesizing or after synthesizing the inorganic support including oxides, nitrides, oxynitrides, chalcogenides, borides, oxyborides, mixtures thereof, or the like of one or two or more elements (M0) selected from alkali metals, transition metals, post-transition metals, and metalloid groups. In addition, in contrast, in a step of synthesizing the inorganic support, synthesis is performed by mixing the materials (raw materials) to be out of the stoichiometric ratio, thereby forming the vacancy defect.

In the case of the inorganic support having the vacancy defect formed by doping and removal of the sacrificial element, the vacancy defect concentration of the inorganic support may be directly controlled by the doping concentration of the sacrificial element, and substantially, the doping concentration of the sacrificial element is the concentration of the vacancy defect included in the support.

The element which may be doped in the substitutional site of the support may be selected based on the size between the element and the doping element positioned in the substitution site and the like, as is known, and a person skilled in the art may properly select the sacrificial element which may be substituted and doped in the sites of one or two or more elements selected from alkali metals, transition metals, post-transition metals, and metalloid group of the inorganic support, considering the spherical material of the inorganic support

As a substantial example based on mesoporous zeolite, which is a representative example of an advantageous inorganic support, an inorganic support having a vacancy defect, specifically a silanol nest formed may be obtained by artificially producing the vacancy defect including the silanol nest by adding a sacrificial element such as boron, indium, aluminum, and/or gallium which is doped in the tetrahedral site in the synthesis step to a synthesis raw material to synthesize an inorganic support and then removing the sacrificial element from the sacrificial element-doped inorganic support by an acid treatment and the like.

As described above, the composite may be used as a catalyst for hydrogenation, dehydrogenation, hydrodesulfurization, hydrodeoxidation, denitrification, or a steam reforming reaction, depending on the known intrinsic properties of each of the spherical alloy materials. In this respect, the manufacturing method of a composite according to the present invention may correspond to a manufacturing method of a catalyst for hydrogenation, dehydrogenation, hydrodesulfurization, hydrodeoxidation, denitrification, or a steam reforming reaction.

### MANUFACTURING EXAMPLE 1

Synthesis of Mesoporous Zeolite having Vacancy Defect Formed

[C₁₈H₃₇₋N⁺(CH₃)₂₋C₆H₁₂—N⁺(CH₃)₂₋O₄H₉][BR⁻]₂ (hereinafter, collectively referred to as C18-6-4) was used as a structural indicator, sodium silicate (29 wt % SiO₂, Si/Na=1.75, Shinheung Silicate) was used as a silica source, and gallium nitrate was used as a gallium source. First, 20 g of a sodium silicate solution was mixed with 24 g of deionized water to prepare a diluted sodium silicate solution, and a structural indicator solution in which 4.7 g of C₁₈₋₆₋₄ and 36 g of the deionized water were mixed and the diluted sodium silicate solution were mixed to prepare a first mixed solution. The thus-prepared first mixed solution was stirred for 30 minutes, a gallium solution in which 0.52 g of gallium nitrate was dissolved in 15.4 g of deionized water was added to the first mixed solution at once to prepare a second mixed solution, and the second mixed solution was aged by stirring the solution at 60° C. for 6 hours. The second mixed solution which was cooled to room temperature after the aging was vigorously stirred, and 18.7 g of an aqueous sulfuric acid (H₂SO₄) solution at a concentration of 0.86 M was added dropwise. The components (by mole) of the finally prepared solution after adding the sulfuric acid were as follows: 100SiO₂/1Ga₂O₃/7.5C_{18-6-4/}30Na₂O/16H₂SO₄/6000H₂O.

The finally prepared solution was added to a Teflon barrel, and the barrel was charged in a stainless autoclave and rotated at 150° C. for 3 days. Thereafter, the formed solid precipitate was filtered and separated from the solution, was dried in an oven at 100° C. for a day, and was fired under the conditions of flowing air at 580° C. to manufacture Ga-doped mesoporous zeolite (hereinafter, collectively referred to as MZ-Ga).

In FIG. 1, (a) is a drawing illustrating results of powder X-ray diffraction analysis of MZ-Ga, (b) is a transmission electron microscope photograph observing MZ-Ga, (c) shows results of a nitrogen adsorption-desorption test, and (d) is a drawing illustrating a BJH pore size distribution calculated from the nitrogen adsorption-desorption test results. As seen in FIG. 1, it is recognized that crystalline zeolite having a crystalline skeleton having an MFI structure was manufactured, and mesoporous zeolite having mesopores having a BET specific surface area of 660 m²/g, a total pore volume (total pore capacity) per unit mass of 1.17 cm³/g, and an average pore size of 5.2 nm was manufactured. In addition, it was confirmed that a molar ratio of Si/Ga included in the zeolite was 48, by plasma elemental analysis (ICP-AES).

In order to selectively remove gallium from MZ-Ga obtained by firing, 1 g of MZ-Ga was added to 100 ml of an aqueous nitric acid solution having a molar concentration of 13 M, the solution was stirred at 100° C. for 12 hours(nitric acid treatment), mesoporous zeolite was recovered by filtration(filtration), and the recovered mesoporous zeolite was washed with deionized water until the cleaning solution is neutral(washing) was used to repeatedly perform a unit process of nitric acid treatment-filtration-washing twice, and then drying in an oven at 100° C. was performed for a day to manufacture mesoporous zeolite from which gallium was removed (hereinafter, referred to as MZ-deGa).

In FIG. 2, (a) is a drawing illustrating results of powder X-ray diffraction analysis of MZ-deGa, (b) is a transmission electron microscope photograph observing MZ-deGa, (c) shows results of a nitrogen adsorption-desorption test of MZ-deGa, and (d) is a drawing illustrating a BJH pore size distribution calculated from the nitrogen adsorption-desorption test results. As seen in FIG. 2, it is recognized that MZ-deGa had substantially similar crystal structure and porosity to MZ-Ga. The BET specific surface area of MZ-deGa was 640 m²/g, and a total pore volume (total pore capacity) per unit mass was 1.19 cm³/g. In addition, as a result of measuring a molar ratio of Si/Ga included in MZ-deGa by plasma elemental analysis (ICP-AES), the mole ratio of Si/Ga was 598, and it is recognized that most of the gallium was removed by the nitric acid treatment.

As a reference material, mesoporous MFI zeolite including no Ga was manufactured. Specifically, 20 g of a sodium silicate solution (29 wt % SiO₂, Si/Na=1.75, Shinheung Silicate) was mixed with 24 g of deionized water to prepare a diluted sodium silicate solution, and a structural indicator solution in which 4.7 g of C₁₈₋₆₋₄ and 67 g of deionized water were mixed and the diluted sodium silicate solution were mixed to prepare a mixed solution. The prepared mixed solution was vigorously stirred for 10 minutes, and then was aged by stirring the solution at 60° C. for 6 hours. The components (by mole) of the finally prepared solution were as follows: 100SiO₂/7.5C₁₈₋₆₋₄/10Na₂O/6000H₂O. The finally prepared solution was added to a Teflon barrel, and the barrel was charged in a stainless autoclave and rotated at 150° C. for 3 days. Thereafter, the formed solid precipitate was filtered, separated from the solution, washed, dried in an oven at 100° C. for a day, and fired under the conditions of flowing air at 580° C. to manufacture mesoporous zeolite (hereinafter, collectively referred to as MZ). In FIG. 3, (a) is a drawing illustrating results of powder X-ray diffraction analysis of MZ, (b) is a transmission electron microscope photograph of MZ, (c) shows results of a nitrogen adsorption-desorption test of MZ, and (d) is a drawing illustrating a BJH pore size distribution calculated from the nitrogen adsorption-desorption test results. As seen from FIG. 3, the manufactured MZ had a substantially similar pore structure to MZ-Ga, and had a BET specific surface area of 660 m²/g and a total pore volume (total pore capacity) per unit mass of 0.83 cm³/g.

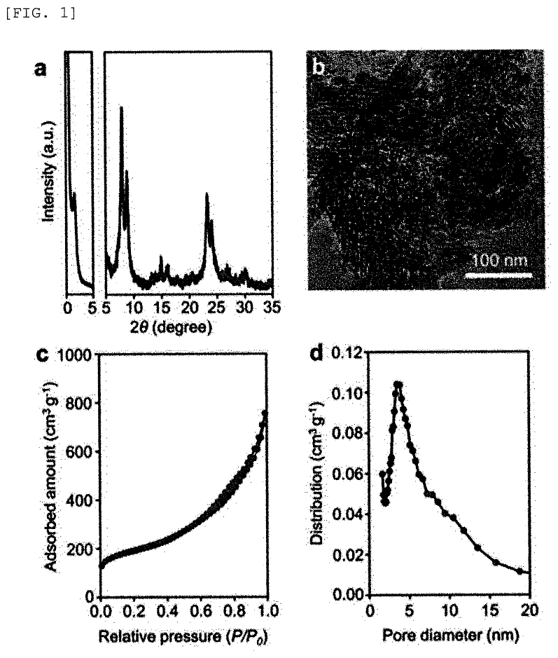

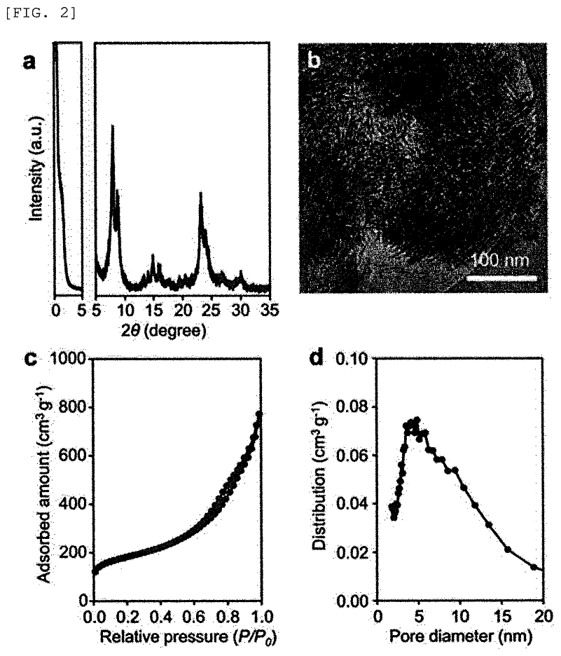

FIG. 4 is a drawing illustrating a spectrum of Fourier-transform infrared spectroscopy (FT-IR) of mesoporous zeolite (MZ), MZ-Ga, and MZ-deGa.

As seen in the FT-IR spectrum of FIG. 4, absorbance bands of Si—OH (about 3750 cm⁻¹) and Ga—OH (about 3600 cm⁻¹) were confirmed in MZ-Ga, and MZ shows one sharp absorbance characteristic corresponding to isolated silanols. However, it was confirmed that in MZ-deGa, a broad absorbance characteristic increased at about 3500 cm⁻¹ indicating a silanol nest was observed.

As is known, the silanol nest refers to a silanol group cluster formed by a plurality of silanol groups being adjacent to the framework defect site of zeolite.

It is recognized from the FT-IR spectrum of FIG. 4 that gallium was removed from MZ-Ga to produce a silanol nest, and thus, MZ-deGa included a silanol nest at a concentration corresponding to the amount of removed gallium.

### EXAMPLES

Platinum and non-platinum metal were introduced to MZ-deGa using an incipient wetness impregnation technique. Pt(NH₃)₄(NO₃)₂ was used as a platinum precursor, and La(NO₃)₃.6H₂O, Y(NO₃)₃.6H₂O, or Ce (NO₃)₃.6H₂O was used as a precursor of a non-platinum metal which was La, Y, or Ce.

The platinum precursor and the precursor of non-platinum metal (La, Y, or Ce) were dissolved in deionized water to prepare a precursor solution, the prepared precursor solution was added to MZ-deGa using an incipient wetness impregnation technique, and drying was performed to introduce platinum and a non-platinum element to MZ-deGa. At this time, the concentrations of the platinum precursor and the non-platinum metal precursor in the precursor solution were adjusted so that MZ-deGa to which a metal (platinum and non-platinum element) was introduced (hereinafter, metal-introduced MZ-deGa) included 1 wt % of platinum and 1 wt % of a non-platinum metal. The metal-introduced MZ-deGa was dried at 60° C. for 6 hours, and the dried metal-introduced MZ-deGa was heat-treated (first heat treatment) at 350° C. for 120 minutes under the atmosphere where pure oxygen flows. The heating rate in the first heat treatment was 0.8° C./min, and the oxygen flow velocity per metal-introduced MZ-deGa unit mass (g_{cat}) was 500 cm³min⁻¹g_{cat}⁻¹.

The first heat-treated MZ-deGa was subjected to a reductive heat treatment (second heat treatment) at 700° C. for 120 minutes under the atmosphere where pure hydrogen flows to manufacture mesoporous zeolite having a platinum-based alloy formed. In the second heat treatment, a hydrogen flow velocity per first heat-treated MZ-deGa unit mass (gnat) was 300 cm³min⁻¹g_{cat}⁻¹. However, when the non-platinum element was Ce, the second heat treatment temperature was 580° C. Hereinafter, the mesoporous zeolite having a platinum-based alloy formed is, when the non-platinum element was Ce, collectively referred to as PtCe-MZ-deGa, when the non-platinum element was La, collectively referred to as PtLa-MZ-deGa, and when the non-platinum element was Y, collectively referred to as PtY-MZ-deGa.

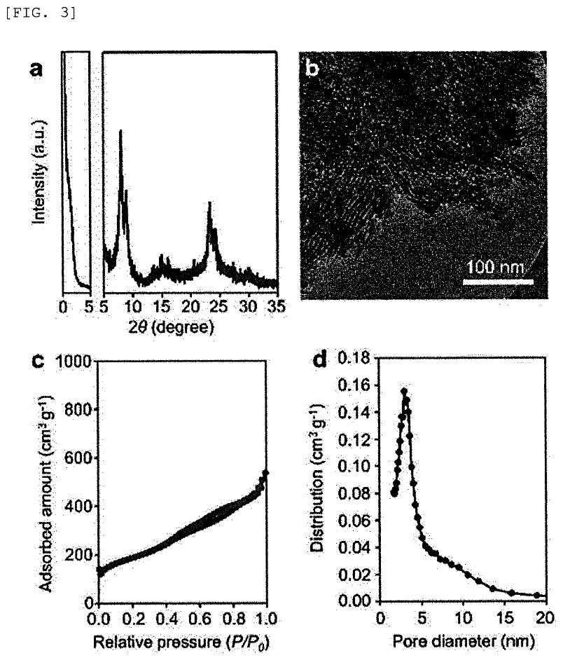

FIG. 5 is high-angle annular dark-field scanning transmission electron microscopy (HAADF-STEM) images observing PtY/MZ-deGa, EDS line profiles, fast Fourier transform (FFT) images, and a schematic diagram illustrating an atomic structure of manufactured Pt—Y alloy.

Specifically, (a) and (b) in FIG. 5 are low magnification HAADF-STEM images of PtY-MZ-deGa. As seen in (a) and (b) in FIG. 5, it is recognized that metal nanoparticles having a uniform size of an average size (diameter) of about 3 nm were dispersed in a MZ-deGa phase. (c) of FIG. 5 is a drawing illustrating EDS line scan profiles analyzed along the arrow in (b) of FIG. 5, and was obtained by measuring signals from a Pt M edge and a Y K edge. It is recognized from (c) of FIG. 5 that both Pt and Y are present in one particle and alloy nanoparticles of Pt—Y were manufactured.

In FIG. 5, (d) is an atomic-magnification HAADF-STEM image with [100] as a zone axis, the inserted image of (d) is a drawing illustrating a structure in which each Y (blue point) column is surrounded by 8 Pt (red point) columns, (e) is a FFT image of (d), (g) is an atomic magnification image HAADF-STEM image with [110] being a zone axis, and (h) is a FFT image of (g). (f) of FIG. 5 is a drawing illustrating a strength profile taken along the <100> direction of a purple arrow in (d). It is recognized from the strength profile of (f) of FIG. 5 that a Pt column and a Y column were alternately positioned in the <100> direction. The results of observing (d) to (h) of FIG. 5 show that since it was consistent with a bulk Pt₃Y L1₂ superlattice structure except a little difference in the atomic distance due to nanocrystallinity, the synthesized nanoparticles were a Pt3Y intermetallic compound having a regular alloy structure of L1₂ superlattice. In addition, a distance and an angle between planes indicated in the atom-magnification HAADF-STEM image were also consistent with the Pt3Y L12 structure. The superlattice reflection in the intermetallic compound structure in (e) and (h) of FIG. 5 is indicated as a yellow arrow. (i) of FIG. 5 is a schematic diagram illustrating an atomic structure of Pt₃Y nanoparticles based on HAADF-STEM and EDS analysis results, in which a grey sphere means Pt and a red sphere means Y.

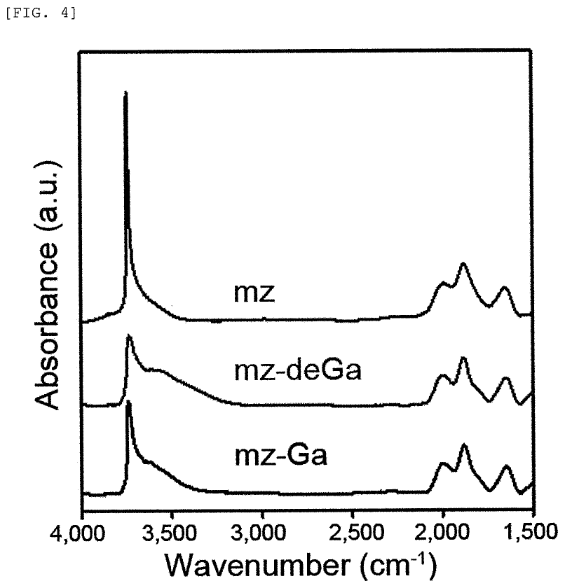

FIG. 6 is high-angle annular dark-field scanning transmission electron microscopy (HAADF-STEM) images observing PtLa-MZ-deGa, an EDS spectrum, and drawings illustrating fast Fourier transform (FFT) images.

Specifically, (a) and (b) of FIG. 6 are low magnification HAADF-STEM images, and it is recognized that nanoparticles having a uniform size (diameter) of about 3 nm on average were dispersed in a MZ-deGa phase. (c) of FIG. 6 is an EDS spectrum in which a region marked with a square in (b) of FIG. 6 was EDS-analyzed, in which it is recognized that both Pt and La were present in one particle. (d) of FIG. 6 is an atom-magnification HAADF-STEM, and (e) of FIG. 6 is a FFT image of (d), in which the superlattice reflection is indicated as a yellow arrow in (d). (f) of FIG. 6 is a drawing illustrating the strength profile of a region indicated as a square dotted line in (d), and it is recognized in (f) of FIG. 6 that a Pt column and a Pt+La column were alternated. From the analysis results of FIG. 6, it is recognized that like the Pt3Y nanoparticles, the manufactured nanoparticles were a Pt3La intermetallic compound having a regular alloy structure of L1₂ superlattice.

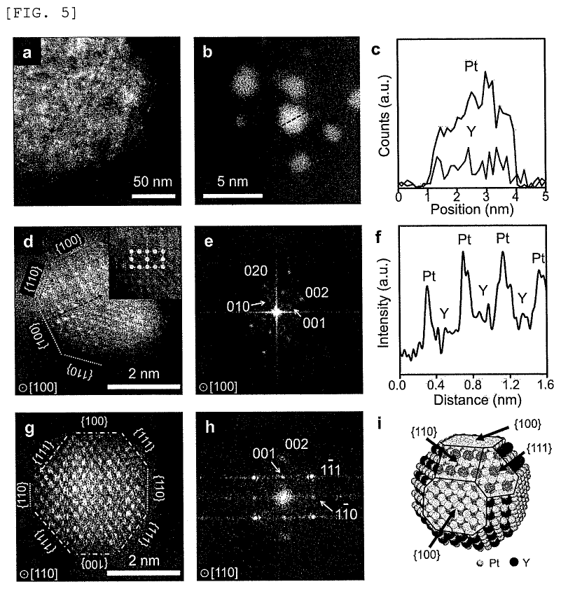

FIG. 7 is high-angle annular dark-field scanning transmission electron microscopy (HAADF-STEM) images observing PtCe-MZ-deGa, an EDS spectrum, and drawings illustrating fast Fourier transform (FFT) images. Specifically, (a) and (b) of FIG. 7 are low-magnification HAADF-STEM images of PtCe-MZ-deGa, and it is recognized that metal nanoparticles having a uniform size were dispersed in MZ-deGa. (c) of FIG. 7 is an EDS spectrum in which a region marked with a square in (b) of FIG. 7 was EDS-analyzed, from which it is recognized that both Pt and Ce were present in one particle. (d) and (g) of FIG. 7 are atom-magnification HAADF-STEM images, and (e) and (h) of FIG. 7 are FTT images of (d) and (g), in which the superlattice reflection in the FFT image is indicated as a yellow arrow. (f) and (i) of FIG. 7 are drawings illustrating the strength profiles of regions indicated as a square dotted line in (d) and (g), and it is recognized from the strength profile that a Pt column and a Pt+Ce column were alternated. From the analysis results of FIG. 7, it is recognized that like the Pt₃Y nanoparticles or Pt₃La nanoparticles, the manufactured nanoparticles were a Pt₃Ce intermetallic compound having a regular alloy structure of L1₂ superlattice.

For comparison, the same method as the method using MZ-deGa as a support was used, but a platinum-based alloy was formed using MZ as a support. However, when the support was MZ, it was confirmed that the non-platinum element and platinum were not alloyed, and only the oxide nanoparticles of the non-platinum element and platinum nanoparticles were formed.

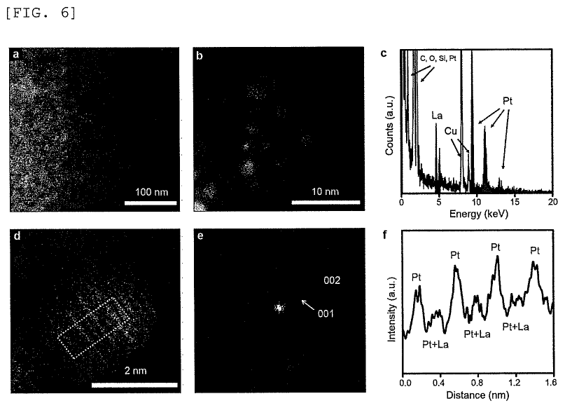

Since a silanol nest which is the largest structural difference between MZ-DeGa and MZ was noted, MZ-deGa to which a non-platinum element was introduced by the incipient wetness impregnation technique and which was subjected to an oxidative heat treatment was observed with HAADF-STEM, which is illustrated in FIG. 8. In FIG. 8, (a) is a low-magnification HAADF-STEM image of MZ-deGa to which La was introduced as a non-platinum element and which was subjected to an oxidative heat treatment, and (b) is the atom-magnification HAADF-STEM image of a square dotted line region of (a). As seen in (a) of FIG. 8, it is recognized that a white dot which may be regarded as a LaOₓ nanoparticle was not observed at all in a zeolite matrix observed in grey. In addition, it was confirmed from (b) of FIG. 8 which is a high magnification atomic image that a La species was dispersed in a single atomic state. (c) of FIG. 8 is an image in which MZ to which a non-platinum element was introduced and which was subjected to an oxidative heat treatment was observed with HAADF-STEM, and it is recognized that when MZ was used as a support, white spots corresponding to LaOₓ nanoparticles were observed.

As seen in FIG. 8, it is recognized that in MZ-deGa having abundant silanol nest, a La species was dispersed single atomically and positioned in the support. Also, from atom-magnification HAADF-STEM observation, it was confirmed that a single atomic species of La showed a rapid and random translational motion on the surface of zeolite (MZ-deGa), and this may be interpreted as due to the fact that the single atomic species of La hopped from one silanol nest to another silanol nest.

From the observation, it is recognized that single atomic diffusion of a rare-earth element species should be premised, for alloying of a rare-earth element with a platinum-based element, in particular, alloying into an intermetallic compound by a reductive heat treatment. Thus, since numerous silanol nests were homogeneously formed in the mesoporous zeolite by removing gallium, it is recognized that the intermetallic compound nanoparticles of Pt₃La and Pt₃Y were homogeneously manufactured over the entire MZ-deGa support.

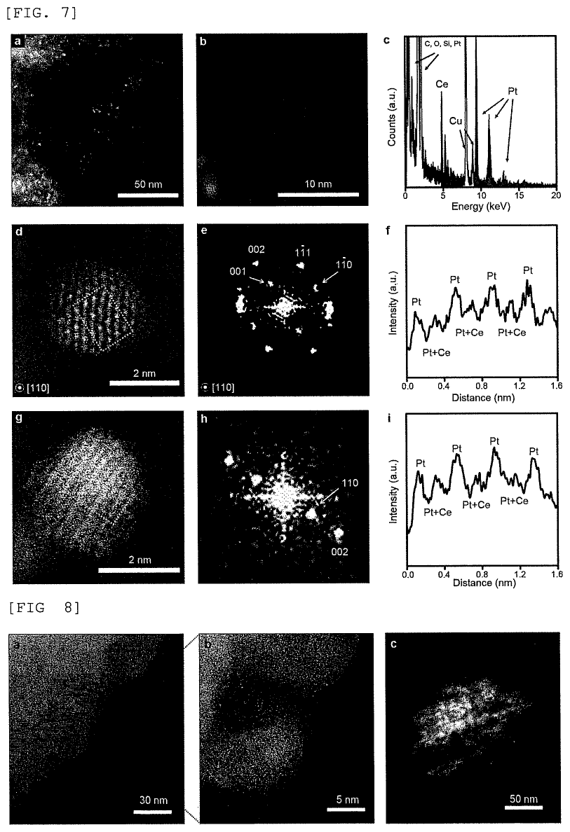

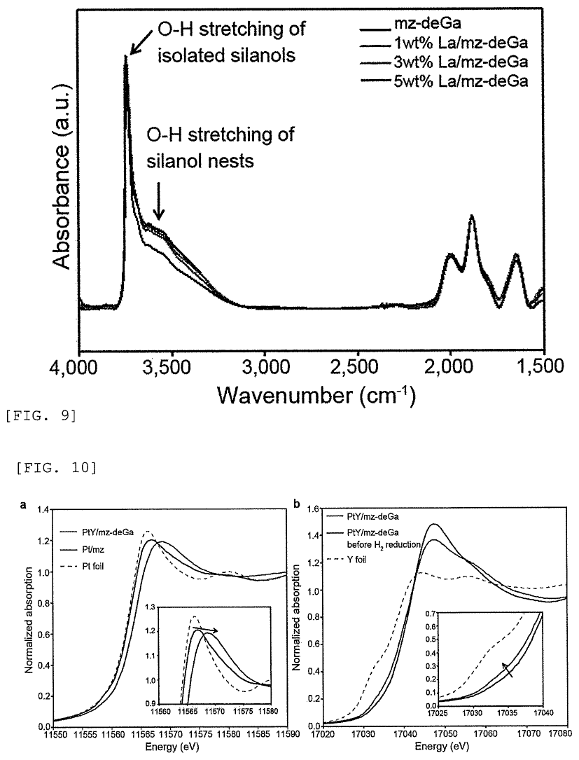

FIG. 9 is a drawing illustrating a FT-IR spectrum of a sample obtained by introducing La at 1 wt %, 3 wt %, or 5 wt % to a mz-deGa support by an incipient wetness impregnation technique and then performing a heat treatment (primary heat treatment) at 350° C. for 120 minutes under an oxygen flow. Here, the spectrum of the support (mz-deGa) is also illustrated for comparison. In FIG. 9, each of the peak (˜3500 cm⁻¹) by the O—H stretching of isolated silanols and the peak by the O—H stretching of the silanol nest is indicated as an arrow. As seen in FIG. 9, it is recognized that as the amount of La introduced to the support was increased, the absorption peak intensity of the silanol nest became gradually weak. This means that La introduced to the support formed a bond with the silanol nest.

It is recognized therefrom that the silanol nest of mesoporous zeolite distributes the rare-earth element which is extremely difficult to reduce as a single atomic species and activates it, and the single atomically distributed rare-earth element is reduced at last by the activation and is atomically supplied to Pt nanoparticles to form intermetallic compound nanoparticles between platinum and a rare-earth element.

The following Table 1 is a table in which hydrochemical adsorption test results at room temperature (25° C.) are summarized.

In Table 1, wt % of Pt, La, and Y refers to wt % of each metal introduced to the support (MZ, MZ-deGa, or alumina) by the incipient wetness impregnation technique. After the designed amounts of platinum and non-platinum element were introduced to the support, alloying was derived in the same manner (first heat treatment and second heat treatment) as described above. In Table 1, H/Ptᵢₙᵢ refers to the number of moles hydrogen which was initially (first) adsorbed per 1 mol of Pt, and H/Pt_{2nd} refers to the number of moles of hydrogen which was adsorbed again per 1 mol of Pt when the initially hydrogen-adsorbed sample was desorbed at room temperature for 1 hour and then hydrogen was adsorbed again. The adsorption was performed by exposing 0.20 g of a sample to a pure hydrogen atmosphere of about 50 torr pressure for 1 hour under a room temperature condition, and a physical adsorption degree was measured while a hydrogen pressure was raised up to 300 torr and a y-intercept value was measured from the chemically adsorbed amount. The desorption was performed by treating the initially hydrogen-adsorbed sample with an ultralow pressure of about 2 mtorr for 1 hour under a 25° C. condition and repeating the measurement process described above.

TABLE 1

| | Pt | La | Y | H/ | H/ | (H/Pt_{2nd})/ |
|---|---|---|---|---|---|---|
| Sample | (wt %) | (wt %) | (wt %) | Ptᵢₙᵢ | Pt_{2nd} | (H/Ptᵢₙᵢ) |
| Pt/mz | 1 | | | 1.01 | 0.36 | 0.36 |
| PtLa/ | 1 | 1 | | 0.5 | 0.5 | 1 |
| mz-deGa | | | | | | |
| PtLa/mz | 1 | 1 | | 0.86 | 0.32 | 0.37 |
| PtLa/ | 1 | 1 | | 0.48 | 0.22 | 0.46 |
| alumina | | | | | | |
| PtY/ | 1 | | 1 | 0.47 | 0.45 | 0.96 |
| mz-deGa | | | | | | |
| PtY/mz | 1 | | 1 | 0.83 | 0.25 | 0.30 |
| PtY/ | i | | 1 | 0.57 | 0.22 | 0.39 |
| alumina | | | | | | |

As seen in Table 1, it is recognized that in all samples except PtLa-MZ-deGa and PtY-MZ-deGa, a significant portion (60-70%) of initially adsorbed hydrogen was not desorbed at room temperature within 1 hour, and thus, H/Pt_{2nd} became much lower than H/Ptᵢₙᵢ. The irreversible hydrogen adsorption as such means that a strong chemical adsorption bond between Pt and H was formed. However, in PtLa-MZ-deGa and PtY-MZ-deGa, a second hydrogen absorption amount taken after initial hydrogen absorption measurement was the same as the initial hydrogen absorption amount, and completely reversible hydrogen chemical adsorption behavior was shown. Since (H/Pt_{2nd})/(H/Ptᵢₙᵢ) of Pt/Mz was only 0.36, completely reversible hydrogen adsorption behavior means that substantially all Pt present in PtLa-MZ-deGa and PtY-MZ-deGa samples were present as an intermetallic compound of Pt₃La and Pt₃Y. Since substantially all Pt introduced to the MZ-deGa support was alloyed into the intermetallic compound of Pt₃M (M=non-platinum element), it may be interpreted that the rest of non-platinum element which was not alloyed among the introduced non-platinum element (La, Y, Ce) remained in the zeolite support as a single atomic species, in the state of being separated from Pt.

Together with the reversibility of hydrogen adsorption, it is recognized that PtLa-MZ-deGa and PtY-MZ-deGa showed hydrogen adsorption of about 0.5 H per Pt, but Pt/Mz as a standard showed hydrogen adsorption of about 1 H per Pt. From the difference, it is recognized that the electronic state of the intermetallic compound was significantly different from the electronic state of pure Pt nanoparticles (Pt/MZ), which was confirmed by X-ray absorption near edge structure (XANES) analysis.

FIG. 10 is drawings illustrating results of analyzing XANES of PtY-MZ-deGa, in which (a) is an XANES spectrum at a PtL₃ edge and illustrates a PtY-MZ-deGa spectrum, a Pt/MZ spectrum for comparison, and a Pt foil spectrum together, and (b) is an XANES spectrum at a Y K edge and is a drawing illustrating a PtY-MZ-deGa spectrum, a spectrum of a sample (PtY/mz-deGa before reduction of (b) of FIG. 10) in the state of being subjected to the first heat treatment (oxidative heat treatment) after introducing Pt and Y to MZ-deGa, that is, in the state before the reductive heat treatment (second heat treatment), and a Y foil spectrum together.

As seen in (a) of FIG. 10, it is recognized that in PtY-MZ-deGa, edge energy and a white line region were greatly moved to higher energy as compared with the Pt/MZ sample including pure Pt nanoparticles. This means that electron donation occurred from a Y metal to a Pt metal neighboring in the bimetal alloy nanoparticles of PtY-MZ-deGa. In addition, as seen in (b) of FIG. 9, it is recognized that edge energy was greatly moved to lower energy after the reductive heat treatment (second heat treatment), and this means that a significant portion of oxidic Y was reduced to metal Y.

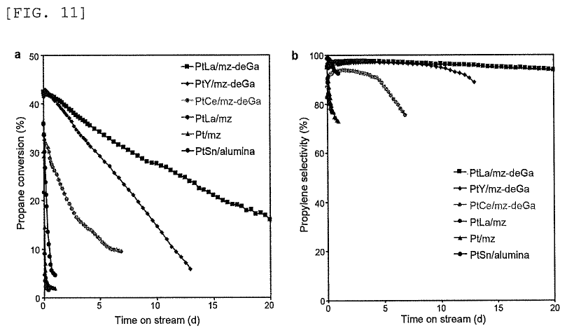

FIG. 11 is a drawing illustrating results of testing propane dehydrogenation catalytic capacities of manufactured PtLa/MZ-deGa, PtY/MZ-deGa, and PtCe/MZ-deGa. For comparison, test results of the catalytic capacity of PtLa/MZ manufactured in the same manner as in the example using MZ as a support, the catalytic capacity of Pt/MZ manufactured in the same manner as in the example except that 1 wt % of Pt was introduced alone by the incipient wetness impregnation technique and the second heat treatment was performed at 580° C., and the catalytic capacity of PtSn/alumina which is a representative example of a commercial catalyst in the conventional dehydrogenation reaction of propane are illustrated together. At this time, PtSn/alumina was manufactured in the same manner as in the example except that the incipient wetness impregnation technique using H₂PtCl₆ and SnCl₁₂.2H₂O as the Pt precursor and the Sn precursor was used to introduce 1 wt % of Pt and 1 wt % of Sn to the alumina support (Sasol, PURALOX γ-A1203, 98%, BET specific surface area=170 m²g⁻¹) and then the second heat treatment was performed at 580° C.

In the propane dehydrogenation reaction, each test sample (catalyst) manufactured was compressed, sieved, and granulated into particles having a diameter of 150 to 350 pm. 50 mg of the granulated test sample was loaded on a quartz tube reactor (diameter: 8 mm) of a fixed-catalyst bed device, the remaining space of the reactor was filled with quartz sand, and the catalyst was loaded. Thereafter, activation was performed at 700° C. (PtLa/MZ-deGa, PtY/MZ-deGa) or 580° C. (Pt/MZ, PtCe/MZ-deGa) for 2 hours in a hydrogen flowing atmosphere of 200 cm³min⁻¹g_{cat}⁻¹ and then a treatment was performed at 580° C. for 1 hour under a purging gas (N₂) flow of 200 cm³min⁻¹g_{cat}⁻¹ to remove chemically adsorbed hydrogen. After N₂ purging, 50 mg of a test sample (catalyst) and pure propane gas (only) were injected, and a dehydrogenation reaction was performed at a weight hourly space velocity (WHSV) of 11 h⁻¹ and 580° C. At this time, on-line gas chromatography which was connected to a catalyst reactor and provided with GS-Gaspro column and a flame-ionization detector (FID) was used to periodically analyze the product.

A propane conversion rate and a propylene selectivity were determined by carbon in each component which was determined by an FID peak area in a discharge product stream. That is, they were calculated by the following equation: propane conversion rate (%)=100*(1−propane carbon/total carbon), propylene selectivity (%)=100*[propylene carbon/(total carbon−propane carbon)]. After propane dehydrogenation test, quartz sand and catalyst particles loaded on the reactor were collected, and coke precipitate amount was analyzed by thermogravimetric analysis. As a result, it was confirmed that coke formation was insignificant, and the distribution of the component may be completely determined by the FID peak.

As seen in FIG. 11, it is recognized that the catalytic performance of the samples manufactured in the examples was significantly improved in all terms including activity, selectivity, and durability. In particular, it is recognized that even under the very harsh reaction conditions where the conventional PtSn/alumina catalyst which is a representative commercialized catalyst was rapidly deactivated within 1 day by severe coke formation, the PtLa/MZ-deGa catalyst having Pt₃La intermetallic nanoparticles formed showed a high initial propane conversion rate of 40% or more (close to equilibrium conversion). In addition, the sample of PtLa/MZ-deGa maintained a conversion rate of 15% or more on the 20th day of the reaction, and maintained a conversion rate of 8% even on 30th day of the reaction. In the Pt/LaMZ sample in which Pt and La were supported on MZ, it is recognized that the initial propane conversion rate was only 22%, and the conversion rate was rapidly decreased to less than 5% within 1.5 hours of the reaction. It is recognized therefrom that a catalyst life was greatly changed to about 3 orders even in the case of the support (MZ, MZ-deGa) having the same MFI framework structure and substantially the same pore structure. In addition, the catalyst performance of PtLa/mz did not show a significant difference from the catalyst performance of Pt/mz, and it is recognized that PtLa/mz was present totally as a monometal nanoparticles without forming an alloy of Pt and La. Like the PtLa/MZ-deGa catalyst, PtY/MZ-deGa also showed a high initial propane conversion rate and propylene selectivity, and a very slow catalyst deactivation. The catalytic capacity and durability of the PtLa/MZ-deGa catalyst were better than those of PtY/MZ-deGa, and this may be interpreted as being due to the difference in atomic size and electronegativity. Specifically, it may be interpreted as being that since La has a larger atomic size and a low electronegativity than Y, the geometric and electronic properties of Pt were more modified.

The significantly improved catalytic capacity and durability of the catalyst including the mesoporous zeolite support and the intermetallic compound nanoparticles of rare-earth element-Pt are based on the role of the silanol nest which may distribute the rare-earth element which is extremely difficult to reduce in the mesoporous zeolite skeleton as a single atomic species and activate it to reduce the rare-earth element by a hydrogen heat treatment and atomically alloy with the Pt nanoparticles. The role of the silanol nest acts identically on other elements which are difficult to reduce, and thus, by introducing metals to be alloyed to the support having the silanol nest formed in a large amount, elements which are difficult to reduce and alloy with low reduction energy may be alloyed.

Hereinabove, although the present invention has been described by specific matters, exemplary embodiments, and drawings, they have been provided only for assisting in the entire understanding of the present invention. Therefore, the present invention is not limited to the exemplary embodiments. Various modifications and changes may be made by those skilled in the art to which the present invention pertains from this description.

Therefore, the spirit of the present invention should not be limited to the above-described exemplary embodiments, and the following claims as well as all modified equally or equivalently to the claims are intended to fall within the scope and spirit of the invention.

1. A composite comprising: a mesoporous inorganic support having a vacancy defect; and metal alloy nanoparticles which are dispersed in and bound to the mesoporous inorganic support and include a precious metal element and a rare-earth element.

2. The composite of claim 1, wherein the support is mesoporous zeolite having the vacancy defect on a mesopore surface.

3. The composite of claim 2, wherein the vacancy defect includes a silanol nest.

4. The composite of claim 2, wherein the support has a vacancy defect concentration of 0.1 mmolg⁻¹ to 1.0 mmolg⁻¹.

5. The composite of claim 1, wherein the precious metal element is one or two or more selected from rhodium (Rh), iridium (Ir), palladium (Pd), platinum (Pt), and ruthenium.

6. The composite of claim 1, wherein the rare-earth element has a standard reduction potential of −2.0 to −3.0 V.

7. The composite of claim 6, wherein the rare-earth element is one or two or more selected from Pr, Sc, Ho, Yb, Dy, Tm, Pm, Gd, Tb, Lu, Sm, Nd, Er, Ce, Y, and La.

8. The composite of claim 1, wherein the metal alloy is an intermetallic compound.

9. The composite of claim 8, wherein the intermetallic compound satisfies the following Chemical Formula 1:
MaₙMbₖ (Chemical Formula 1)
wherein Ma is a precious metal element, Mb is a rare-earth element, n is an integer of 1 to 7, and k is an integer of 1 to 3.

10. The composite of claim 1, wherein the nanoparticles have an average diameter of 1 to 5 nm.

11. The composite of claim 10, wherein an average separation distance between the nanoparticles which are dispersed in and bound to the support is 1 nm to 10 nm.

12. The composite of claim 1, wherein the composite includes 0.5 to 5.0 wt % of the nanoparticles.

13. A catalyst comprising the composite of claim 1.

14. The catalyst of claim 13, wherein the catalyst is for hydrogenation, dehydrogenation, hydrodesulfurization, hydrodeoxidation, denitrification, or a steam reforming reaction.

15. A method of dehydrogenating a saturated hydrocarbon raw material using a catalyst including the composite of claim 1.

16. The method of claim 15, wherein the saturated hydrogenation raw material includes propane.

17. A manufacturing method of a composite, the method comprising:
a) introducing a precious metal element and a rare-earth element to a mesoporous inorganic support having a vacancy defect using an impregnation method;
b) subjecting the support to which the precious metal element and the rare-earth element are introduced to an oxidative heat treatment; and
c) subjecting the oxidatively heat-treated support to a reductive heat treatment.

18. The manufacturing method of a composite of claim 17, wherein the mesoporous inorganic support having a vacancy defect of a) is mesoporous zeolite from which a sacrificial element doped in a substitutional site is removed and which has a vacancy defect.

19. The manufacturing method of a composite of claim 18, wherein a concentration of the vacancy defect is controlled by a doping concentration of a sacrificial element.

20. The manufacturing method of a composite of claim 19, wherein the sacrificial element is one or more selected from aluminum, gallium, indium, and boron.

21. The manufacturing method of a composite of claim 18, wherein before c), the rare-earth element is dispersed in an atomic unit and positioned in the vacancy defect of the support.

22. The manufacturing method of a composite of claim 18, wherein the oxidative heat treatment is performed at 300 to 500° C. under an oxygen flow.

23. The manufacturing method of a composite of claim 18, wherein the reductive heat treatment is performed at 300 to 750° C. under a hydrogen flow.

24. The manufacturing method of a composite of claim 17, wherein in a), 0.5 to 3 wt % of each of the precious metal element and the rare-earth element is introduced.
